(19) United States
(12) Patent Application Publication
(10) Pub. No.: US 2007/0038272 A1
(43) Pub. Date: Feb. 15, 2007

(54) Toothbrush

(76) Inventor: Wan-Chi Liu, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/199,072

(22) Filed: Aug. 9, 2005

(51) Int. Cl. A61N 5/06; A61C 1/00; A61C 3/00

(52) U.S. Cl. 607/88; 433/29

(57) ABSTRACT

A toothbrush includes a head capable of producing a certain wavelength of light such that when the user is brushing teeth, the light from the head will kill bacteria, enhance blood circulation, vitalize tissues, improve tissue reproduction and repair, and reduce the risk of oral diseases and wound infection in his mouth.

### BACKGROUND OF THE INVENTION

1. Field of the invention

The present invention relates to a toothbrush, more particularly one, of which the head can produce a certain wavelength of light such that when the user is brushing teeth, light will be produced to kill bacteria, enhance blood circulation, vitalize tissues, improve tissue reproduction and repair, and reduce the risk of oral diseases and wound infection in his mouth, and such that after the toothbrush is used, light will be produced to kill bacteria on the bristles, thus preventing bacteria from rapidly breeding on the head of the toothbrush.

2. Brief Description of the Prior Art

After eating, there will be much food residue in the mouth, which is a good source of nutrition for bacteria. And, if teeth are left unbrushed, plaque will begin to accumulate within three minutes after eating. One milligram of plaque contains more than one hundred million bacteria, and bacteria are the major cause of oral diseases such as dental caries, tartar, periodontitis, etc. Therefore, the most effective way to prevent dental caries, tartar, and periodontitis is to brush teeth immediately after eating.

Toothbrushes are the major tools for cleaning teeth. If a toothbrush isn't properly cleaned after use, it is prone to breed bacteria, which will cause oral diseases and digestive system diseases when the toothbrush is used again.

### SUMMARY OF THE INVENTION

It is a main object of the invention to provide an improvement on a toothbrush to overcome the above-mentioned problem.

The toothbrush of the present invention includes a head capable of producing a certain wavelength of light. Thus, when the user is brushing teeth, the light from the head of the toothbrush will kill bacteria, enhance blood circulation, vitalize tissues, improve tissue reproduction and repair, and reduce the risk of oral diseases and wound infection in his mouth. And, after the toothbrush is used, the light will kill bacteria on the bristles, thus preventing bacteria from rapidly breeding on the head of the toothbrush.

### BRIEF DESCRIPTION OF THE DRAWINGS

The present invention will be better understood by referring to the accompanying drawings, wherein:

FIG. 1 is a front view of the first preferred embodiment of a toothbrush in the present invention,

FIG. 2 is a side view of the first preferred embodiment,

FIG. 3 is a side view of the second preferred embodiment,

FIG. 4 is a partial side view of the third preferred embodiment, and

FIG. 5 is a partial side view of the fourth preferred embodiment.

### DETAILED DESCRIPTION OF THE PREFERRED EMBODIMENTS

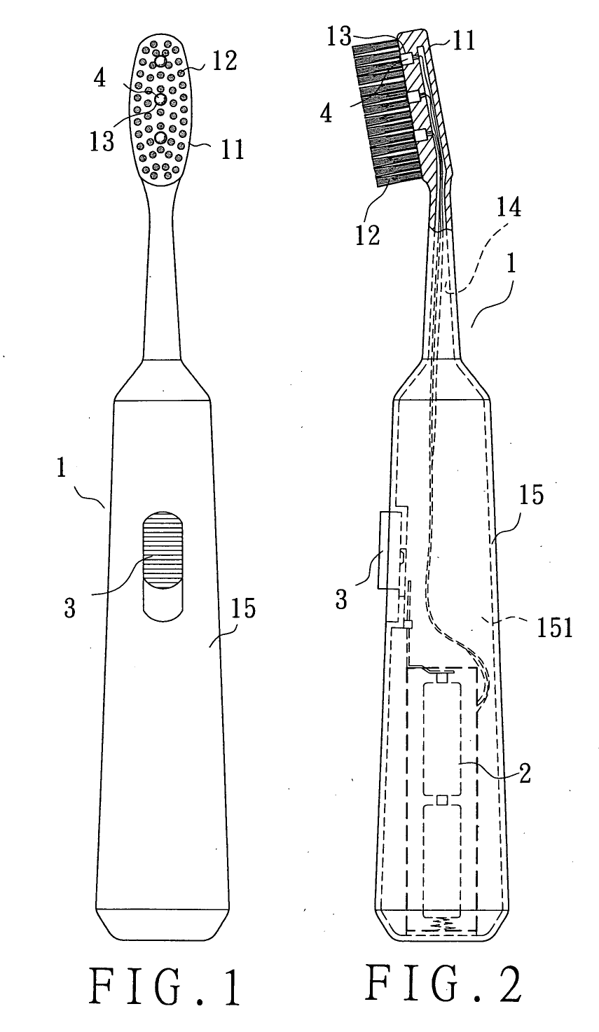

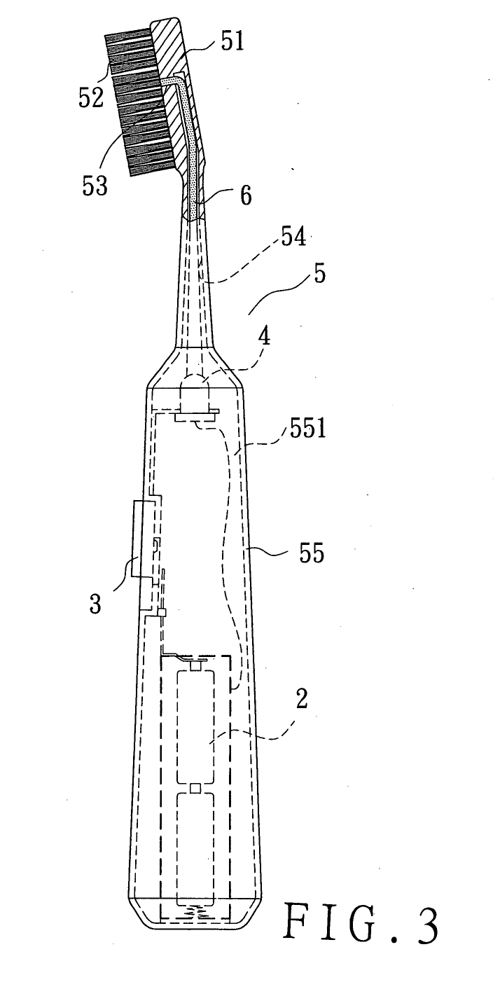

Referring to FIGS. 1 and 2, a first preferred embodiment of a toothbrush 1 includes a head 11, bristles 12 on a first side of the head 11, a handle 15, a power source unit 2, a switch 3, and several light producing elements 4. The head 11 has several through holes 13 on the first side thereof, and a holding room 14 therein, which communicates with a holding room 151 of the handle 15. The light producing elements 4 are held in the through holes 13 of the head 11, and they can produce a certain wavelength of light such as infrared, visible light, ultraviolet light, and laser light. The power source unit 2 is held in the handle 15 while the switch 3 is positioned on an outer side of the handle 15 for controlling electricity from the power source unit 2. And, the power source unit 2, the switch 3, and the light producing elements 4 are connected to form a circuit. Therefore, light will travel outside the head 11 when the light producing elements 4 are powered.

Referring to FIG. 3, a second preferred embodiment of a toothbrush 2 includes a head 51, bristles 52 on a first side of the head 51, a handle 55, a power source unit 2, a switch 3, a light producing element 4, and a light guiding member 6. The head 51 has a through hole 13 on the first side thereof, and a holding room 54 therein, which communicates with a holding room 551 of the handle 55. The light producing element 4 is held in the holding room 54 of the head 51, and they can produce a certain wavelength of light such as infrared, visible light, ultraviolet light, and laser light. The power source unit 2 is held in the handle 55. The switch 3 is positioned on an outer side of the handle 15 for controlling electricity from the power source unit 2. One end of the light guiding member 6 is next to a light emitting end of the light producing element 4, and the other end of the light guiding member 6 is fitted in the through hole 53 of the head 51. And, the power source unit 2, the switch 3, and the light producing elements 4 are connected to form a circuit. Therefore, light will travel outside the head 51 owing to guidance of the light guiding member 6 when the light producing elements 4 are powered.

Therefore, when the user is brushing his teeth with one of the above toothbrushes, light emitted from the light producing elements 4 will kill bacteria in his oral cavity, reducing the risk of oral diseases and wound infection in his mouth.

The light producing elements 4 can be the kind that produces infrared light, which is invisible, and which will produce heat to make the user feel warm, enhance blood circulation, vitalize tissues, and improve tissue reproduction and repair.

The light producing elements 4 can be the kind that produces visible light, e.g. blue light, and green light, which will cause chemical reaction to produce certain effects.

The light producing elements 4 can be the kind that produces ultraviolet light, which can effectively bacteria and viruses without causing side effects, corrosion, pollution or residues.

Or, the light producing elements 4 can be the kind that produces laser light, which is concentrated and intensified, and can kill bacteria; there are various wavelengths of laser light, which will cause different chemical reactions.

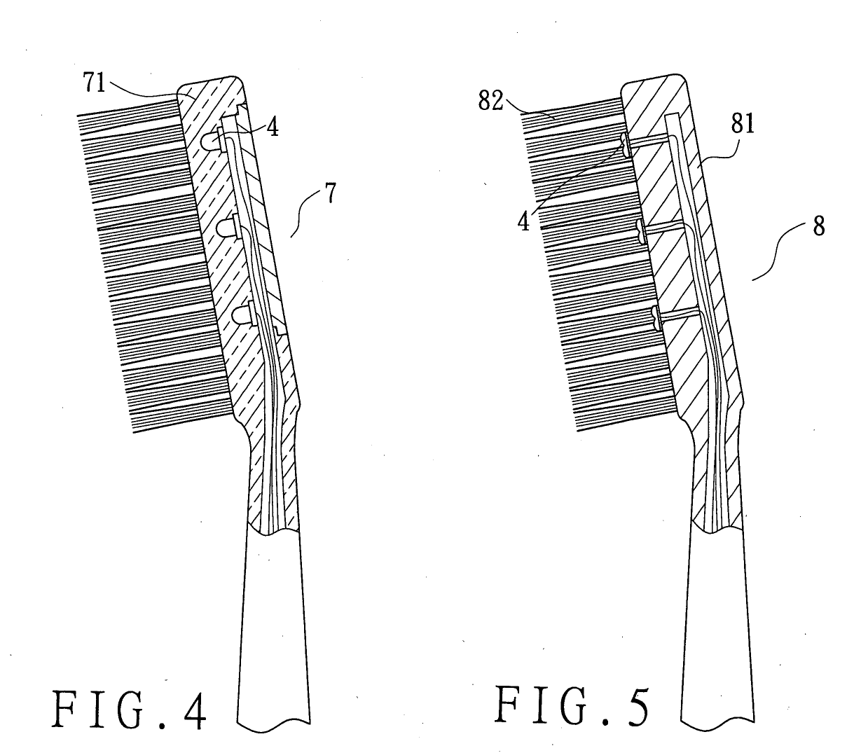

Referring to FIG. 4, a third preferred embodiment of a toothbrush 7 includes a head 71 made of a material pervious to light, light producing elements 4 held inside the head 71, a power source unit 2, and a switch 3 for controlling electricity from the power source unit 2; the light producing element 4 can produce a certain wavelength of light such as infrared, visible light, ultraviolet light, and laser light; the power source unit 2, the switch 3, and the light producing elements 4 are connected to form a circuit.

Referring to FIG. 5, a fourth preferred embodiment of a toothbrush 8 includes a head 81, bristles 82 on a first side of the head 81, light producing elements 4 positioned on the first side of the head 81, a power source unit 2, and a switch 3 for controlling electricity from the power source unit 2; the light producing element 4 can produce a certain wavelength of light such as infrared, visible light, ultraviolet light, and laser light; the power source unit 2, the switch 3, and the light producing elements 4 are connected to form a circuit.

From the above description, it can be seen that the toothbrushes of the present invention has the following advantages: when the user is brushing teeth, the light producing elements can be activated to produce light so as to kill bacteria, enhance blood circulation, vitalize tissues, improve tissue reproduction and repair, and reduce the risk of oral diseases and wound infection in his mouth; after the toothbrushes are used, the light producing elements can be activated to produce light so as to kill bacteria on the heads and the bristles of the toothbrushes, thus preventing bacteria from breeding rapidly on the heads and the bristles.

What is claimed is:

1. A toothbrush, comprising
a head;
a plurality of light producing elements held in position with the head; the light producing elements being capable of producing a certain wavelength of light;
a power source unit; and
a switch;
the light producing elements, the power source unit, and the switch being connected to form a circuit.

2. The toothbrush as claimed in claim 1, wherein the head has at least one through hole thereon, and the light producing elements are fitted in the through hole of the head.

3. The toothbrush as claimed in claim 1, wherein the light producing elements are positioned on a surface of the head.

4. The toothbrush as claimed in claim 1, wherein the head is made of a material pervious to light, and the light producing elements are held inside the head.

5. A toothbrush, comprising
a head having a through hole thereon; and
a light producing element held in the head; the light producing element being capable of producing a certain wavelength of light, which travels outside through the through hole of the head.

6. The toothbrush as claimed in claim 5 further having a light guiding member; one end of the light guiding member being next to a light emitting end of the light producing element; other end of the light guiding member being fitted in the through hole of the head such that light emitted from the light producing element will travel outside the head owing to guidance of the light guiding member.
